(19) United States
(12) Patent Application Publication
(10) Pub. No.: US 2021/0205489 A1
(43) Pub. Date: Jul. 8, 2021

(54) DIFFUSER PRODUCT AND ITS MANUFACTURING METHOD

(71) Applicant: DONGGUAN YIH TEH ELECTRIC PRODUCTS CO., LTD., DongGuan City (CN)

(72) Inventor: Ming Jen Hsiao, Miaoli County (TW)

(21) Appl. No.: 16/737,460

(22) Filed: Jan. 8, 2020

(51) Int. Cl. A61L 9/12; D21H 27/00; B31D 5/00

(52) U.S. Cl. CPC A61L 9/127; B32B 1/08; B31D 5/0091; D21H 27/007

(57) ABSTRACT

A diffuser product and its manufacturing method is disclosed. The diffuser product includes a absorbent paper structure component, and a breathable film bonded to the outer side of the absorbent paper structure component to shield the absorbent paper structure component in a waterproof and breathable manner and to adjust the aromatic substance to contact with the outside air to further control the release rate of the aromatic molecules evaporated by the aromatic substance through the breathable film and prevent the outside moisture from penetrating into the inside of the absorbent paper structure component, thereby maintaining the quality of the absorbent paper structure component.

### BACKGROUND OF THE INVENTION

### 1. Field of the Invention

The present invention relates to aroma diffuser technology and more particularly, to a diffuser product and its manufacturing method, which adjusts the aromatic substance to contact with the outside air to further control the release rate of the aromatic molecules evaporated by the aromatic substance.

### 2. Description of the Related Art

Diffuser products are used to absorb aromatic substances and diffuse the absorbed aromatic substances into the surrounding atmosphere. The matrix material of these products or the product fibers of their structural parts contain pores, and the product fibers or bodies of the aforementioned matrix materials or their structural parts contain porosity, which can affect the aromatic substances absorption rate or the amount of the aromatic substances that can be carried, or it may affect the rate of fragrance release.

In some prior art designs, the aromatic substances that are kept in the pores of the matrix material or the product fibers can be evaporated rapidly. Especially for compositions containing more volatile compounds, such as aromatic products containing top notes, the aromatic substance is released in large quantities and quickly in the early stages of product use. Under the same fragrance loading and characteristics, these prior art diffuser products are difficult to provide long-lasting and stable fragrance release. For this reason, common diffuser products such as diffuser sticks are often coated with a layer of paint. For example, in U.S. Publication No. 2011/02623, it is disclosed that coatings are used to delay volatile substances. However, these coating layers often also absorb volatile substances in the coatings, and the coatings also reduce the pores of the diffuser paper stick to release the volatile substances effectively. Once the concentration of volatile substances inside the product reaches equilibrium, there will no longer be sufficient concentrations to drive the release of volatile substances through the coating.

In addition, the paper or its structural parts formed by the pulp through the manufacturing process usually contains some additional water or capillary water, etc., occupying the pores or surface of the paper or its structural parts of the matrix material, affecting the absorption and loading of aromatic substances.

Furthermore, the paper matrix material or its structural components may be better than ordinary fiber, porous plastics or polymers, gypsum or wood in aromatic substance absorption rate, load factor or release rate, however, the paper material is susceptible to moisture and is not water resistance. The bonding force between the bonds of the matrix material is reduced, and the paper material is easily disintegrated. Especially after the slender paper stick product absorbs liquid aromatic substances, the overall strength of the matrix material is affected.

### SUMMARY OF THE INVENTION

The present invention has been accomplished under the circumstances in view. It is one object of the present invention to provide a diffuser product, which has a waterproof breathable film at the outer side that adjusts the aromatic substance to contact with the outside air to further control the release rate of the aromatic molecules evaporated by the aromatic substance, thereby prolonging fragrance diffusing time and providing fragrance into the space in a stable and effective manner.

It is one object of the present invention to provide a diffuser product, which has a waterproof breathable film with micropores at the outer side. In this way, the diffuser product is shielded from water and air, so that the external moisture will not penetrate into the inside, which will help transportation and storage, maintain the quality of diffuser product, and prevent the aromatic substance from leaking out of the diffuser product.

It is one object of the present invention to provide a diffuser product, which has a breathable film bonded to an outer side of an absorbent paper structure component. The breathable film contains ductility or elasticity, which can allow the diffuser product to strengthen the solidity and increase the mechanical properties such as toughness to improve the quality of use.

To achieve these and other objects of the present invention, a diffuser product comprises an absorbent paper structure component, at least one aromatic substance, and at least one breathable film. The absorbent paper structure component comprises an absorbent paper. The absorbent paper has a plurality of pores, and a plurality of tiny air channels formed between the pores for creating capillary effects. The at least one aromatic substance is releasably filled in the pores of the absorbent paper of the absorbent paper structure component. The at least one breathable film is bonded to an outer side of the absorbent paper or absorbent paper structure component. The at least one breathable film has a plurality of micropores that are waterproof and breathable. Thus, the at least one breathable film shield the absorbent paper structure component in a waterproof and breathable manner, and is used to adjust the at least one aromatic substance to contact with the outside air, thereby controlling the release rate of aromatic molecules evaporated by the at least one aromatic substance through the breathable film and also controlling the outside moisture from penetrating into the inside of the absorbent paper structure component.

Preferably, the breathable film is a microporous film, which is used to restrict the release of some of the aromatic substance to the outside while the micropores of the film allow some of the aromatic substance to gradually diffuse into the environmental space at a stable rate.

Adjusting the release rate of the volatile aromatic substance with a proper amount of diffusion can delay the aroma diffusing time of the diffuser product, provide a stable and effective aroma to the space, and overcome the diffuser product's excessive consumption of the aromatic substance in the early stages of use.

Preferably, the breathable film is a waterproof and moisture-permeable microporous film. With this, the breathable film can shield the absorbent paper structure component in a waterproof and breathable manner and adjust the aromatic substance to contact with the outside air to further control the release rate of the aromatic molecules evaporated by the aromatic substance through the breathable film and prevent the outside moisture from penetrating into the inside of the absorbent paper structure component, thereby maintaining the quality of the absorbent paper structure component.

To achieve these and other objects of the present invention, a diffuser product manufacturing method comprises the steps of: A diffuser product manufacturing method, comprising the steps of (a) placing an absorbent paper structure component or absorbent paper in an oven and then operating the oven to bake the absorbent paper structure component or absorbent paper at about 60° C.-80° C. for about 15-25 minutes, and then removing the baked absorb paper structure component or absorbent paper from the oven; (b) using a container to fill at least one liquid aromatic substance into the absorbent paper structure component or absorbent paper for enabling the at least one liquid aromatic substance to be releasably remained in the pores of the absorbent paper structure component or absorbent paper; (c) removing the absorbent paper structure component or absorbent paper being filled with the at least one liquid aromatic substance from the container for drying so that the dried absorbent paper structure component or absorbent paper is suitable for diffusing the at least one aromatic substance into the atmosphere; and (d) bonding at least one breathable film to the outer side of the dried absorbent paper structure component or absorbent paper.

In this way, the shields the absorbent paper structure component in a waterproof and breathable manner, and is used to adjust the at least one aromatic substance to contact with the outside air, thereby controlling the release rate of aromatic molecules evaporated by the at least one aromatic substance through the breathable film and also controlling the outside moisture from penetrating into the inside of the absorbent paper structure component.

Furthermore, the breathable film is used to gradually diffuse the aromatic substance into the environmental space at a stable rate.

Furthermore, adjusting the release rate of the volatile aromatic substance with a proper amount of diffusion can delay the aroma diffusing time of the diffuser product, provide a stable and effective aroma to the space.

Preferably, the breathable film is a waterproof and moisture-permeable microporous film. With this, the breathable film can shield the absorbent paper structure component in a waterproof and breathable manner and adjust the aromatic substance to contact with the outside air to further control the release rate of the aromatic molecules evaporated by the aromatic substance through the breathable film and prevent the outside moisture from penetrating into the inside of the absorbent paper structure component.

To achieve these and other objects of the present invention, a diffuser product comprises a rolled paper stick an at least one aromatic substance. The rolled paper stick is a multilayer structure formed by wrapping a planar absorbent paper around a central axis. The rolled paper stick is selectively a solid or hollow shaft. The planar paper has a first surface and a second surface, a plurality of pores formed on each of the first surface and the second surface and a plurality of micro air channels formed on each of the first surface and the second surface between the pores for creating capillary effects. The first surface and the second surface have the same porosity. The rolled paper stick is dried so that the pores have high saturation absorptivity. The at least one aromatic substance is releasably filled in the pores of the dried rolled paper stick.

Compared with the conventional rolled paper stick, the rolled paper stick of the present invention has pores that form excellent absorption gradients or excellent fragrance release gradients. The diffuser product of the present invention has high absorption load and good capillary action.

Other advantages and features of the present invention will be fully understood by reference to the following specification in junction with the accompanying drawings, in which like reference signs denote like components of structure.

### BRIEF DESCRIPTION OF THE DRAWINGS

FIG. 1 is an enlarged view of a part of an absorbent paper for diffuser product in accordance with the present invention.

FIG. 2 illustrates the outer appearance of an absorbent paper structure component for diffuser product in accordance with the present invention.

FIG. 3 illustrates the outer appearance of a rolled paper stick of the diffuser product in accordance with the present invention.

FIG. 4 is a schematic drawing illustrating the bonding between the absorbent paper structure component and the breathable film.

FIG. 5 illustrates the breathable film bonded to the planar absorbent paper.

FIG. 6 illustrates the outer appearance of the rolled paper stick with the bonded breathable film.

FIG. 7 is a sectional elevation, in an enlarged scale, of the absorbent paper structure component.

FIG. 8 illustrates the structure of the absorbent paper for diffuser product in accordance with the present invention.

### DETAILED DESCRIPTION OF THE PREFERRED EMBODIMENT

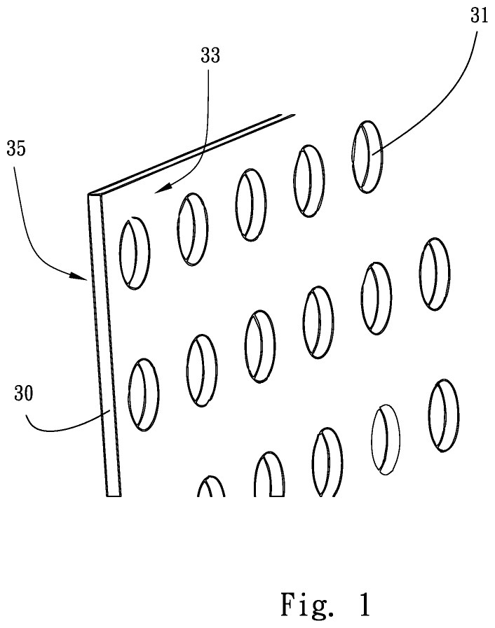

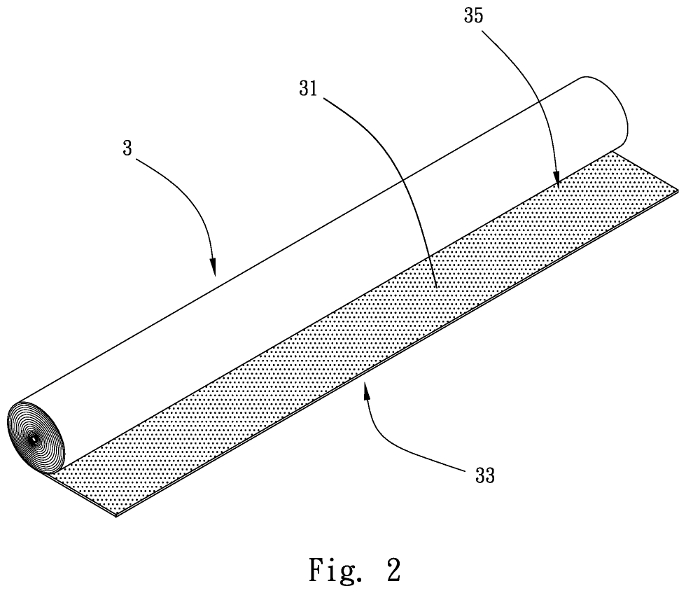

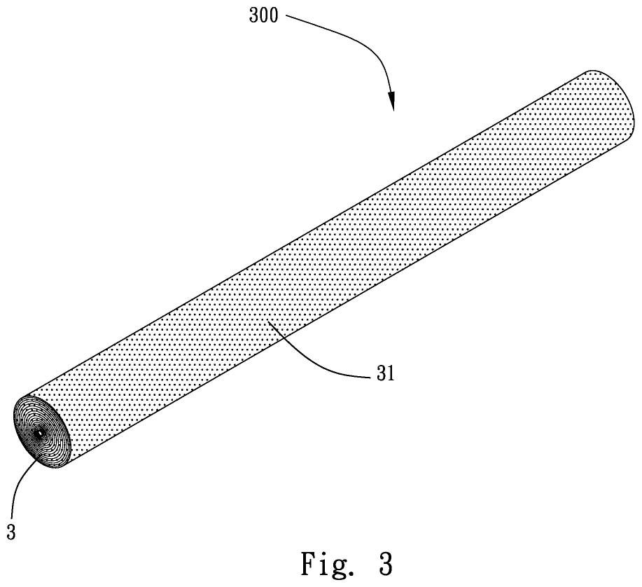

Referring to FIGS. 1-6, a diffuser product in accordance with the present invention comprises an absorbent paper structure component 3, at least one aromatic substance, and at least one breathable film 5. The absorbent paper structure component 3 comprises an absorbent paper 30. The absorbent paper 30 has pores 31, and micro air channels (not shown) formed between the pores 31 for creating capillary effects.

The at least one aromatic substance is releasably filled in the pores 31 of the absorbent paper structure component 3.

The at least one breathable film 5 has a plurality of micropores that are waterproof and breathable. The breathable film 5 is bonded to the outer side of the absorbent paper 30 or the absorbent paper structure component 3.

In this way, the breathable film 5 can shield the absorbent paper structure component 3 in a waterproof and breathable manner, and is used to adjust the aromatic substance to contact with the outside air, thereby controlling the release rate of the aromatic molecules evaporated by the aromatic substance through the breathable film 5 and also controlling the outside moisture from penetrating into the inside of the absorbent paper structure component 3.

The breathable film 5 is a microporous film that is used to restrict the release of some of the aromatic substance to the outside while the micropores of the breathable film 5 allow some of the aromatic substance to gradually diffuse into the environmental space at a stable rate.

By adjusting the release rate of the appropriate amount of the volatile aromatic substance, it can delay the aroma diffusion time of the diffuser product, provide a stable and effective aroma to the space, and overcome the problem that the diffuser product consumes the aromatic substance too quickly in the early stage of use and the problem of insufficient concentration in the later stage.

The breathable film 5 is a waterproof and moisture-permeable microporous film. Therefore, the breathable film 5 not only can shield the absorbent paper structure component 3 in a waterproof and breathable manner and adjust the aromatic substance to contact with the outside air to further control the release rate of the aromatic molecules evaporated by the aromatic substance through the breathable film 5 but also can control the outside moisture from penetrating into the inside of the absorbent paper structure component 3 to maintain the quality of the absorbent paper structure component 3.

The matrix material of the absorbent paper 30 of the absorbent paper structure component 3 includes, but is not limited to, for example, pulp, pulp compounds, pulp compositions, fibers, and etc. Pulp may include fibers and raw materials but is not limited to wood fibers, and other plant fibers such as roots, stems or leaves of various herbs, manuscript rods, hemp, cotton, bagasse or peel fibers. This matrix material can form the absorbent paper 30 of the absorbent paper structure component 3.

The aromatic substance includes an aromatic odor composition, a volatile substance, or a liquid aromatic substance, such as a fragrance or fragrance composition, an aromatherapy compound, a water-based fragrance, or a natural oil, etc. These include volatile substances that can be transmitted to the olfactory system and can include one or more aromatic characteristics, such as fragrance ingredients such as top notes, middle notes or base notes. These fragrance ingredients or their compositions are often used, such as various raw materials or compositions including but not limited to essential oils, aromatic essential oils, various flavors, flower essences, gels or sesame oils or fragrances, or their liquid aromatic substances. Those skilled in the art can know the use.

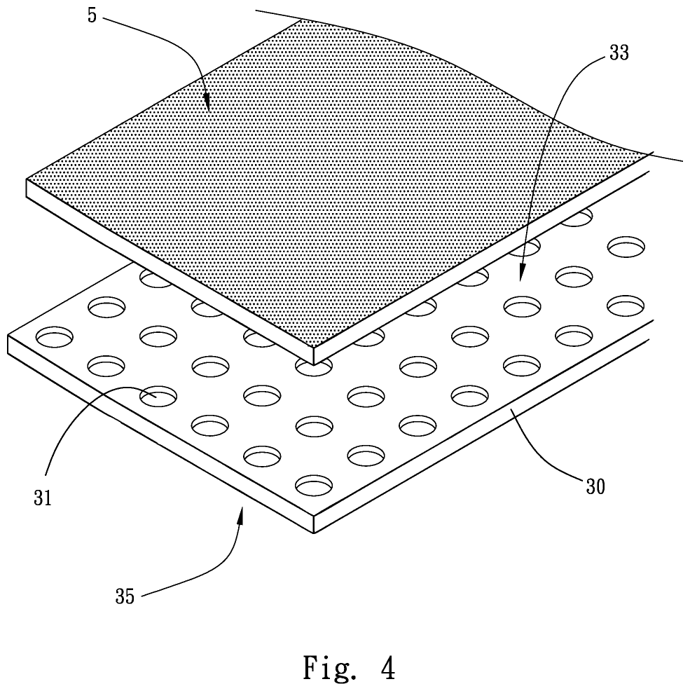

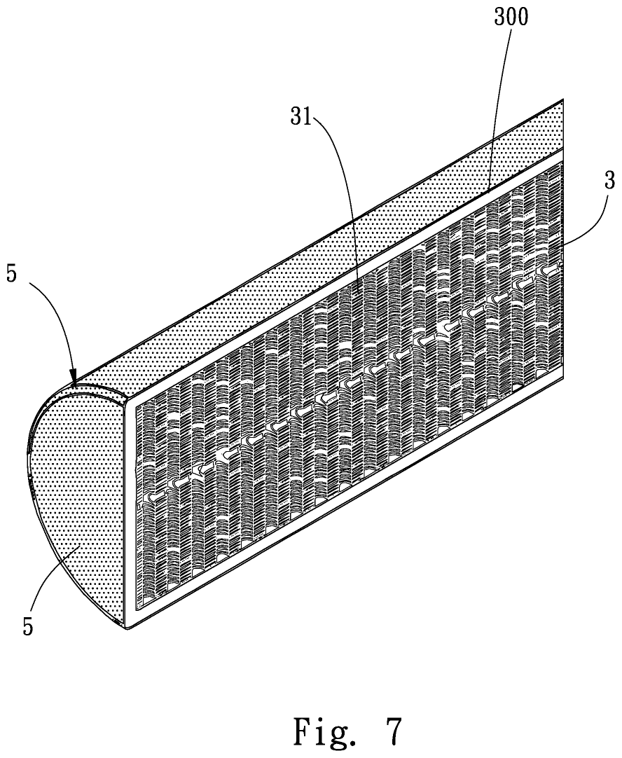

Referring to FIGS. 4-7, according to some embodiments of the present invention, the breathable film 5 is made from thermoplastic polyurethane (TPU), polytetrafluoroethylene (TPFE), polyethylene, polypropylene, or ultra-high molecular weight polyethylene, polytetrafluoroethylene or any of microporous films containing pores-forming compounds such as foaming agents. In addition to the breathable and waterproof properties, in some embodiments, the film properties themselves can be attached or adsorbed or coated and bonded to the outer side of the absorbent paper structure component 3 or absorbent paper 30. By bonding the breathable film 5 to the outer side of the absorbent paper 30 or absorbent paper structure component 3, the ductility or elasticity of the breathable film 5 can allow the diffuser product to strengthen the solidity and increase the mechanical properties such as toughness, thereby improving the quality of use.

Referring to FIGS. 4-7, according to some embodiments of the present invention, the breathable film 5 is bonded to at least a part or all or the end of the outer side of the absorbent paper 30 or absorbent paper structure component 3, or it is bonded with the outer side of the absorbent paper structure component 3 at intervals.

Referring to FIGS. 1-6, according to some embodiments of the present invention, the absorbent paper structure component 3 can be, but is not limited to, a paper roll, a paper stick, a paper piece, a paper block, a two-dimensional paper product, a three-dimensional paper product or a geometrically shaped absorbent paper structure component. The absorbent paper structure component 3 can be, but is not limited to, a paper stick, and the paper stick can be a paper roll or a single structural paper stick made by casting. In some embodiments, the absorbent paper 30 has a first surface 33 and a second surface 35. The first surface 33 and the second surface 35 have the same porosity, the same number of pores, or the same pore size. An absorption gradient is formed between the layers, so that the absorbency of the absorbent paper structure component 3 increases from the inner layer to the outer layer, or the absorbency decreases from the inner layer to the outer layer. This absorbent paper structure component 3 absorbs aromatic substances, such as liquid aromatic substance essences or essential oils or sesame oil. The outer layer absorbs more than the inner layer and can smoothly fill the fragrance to the inner layer or release the aromatics without hindrance. The product manufacturing process can be efficient and rapid.

Referring to FIGS. 1-3 and FIGS. 6 and 7, in some embodiments of the present invention, the absorbent paper structure component 3 is a rolled paper stick 300. The rolled paper stick 300 is a multilayer structure formed by wrapping a planar paper around a central axis. The rolled paper stick 300 can be a solid or hollow shaft. The planar paper has a first surface 33 and a second surface 35. The first surface 33 and the second surface 35 have the same porosity, the same number of pores, or the same pore size. There is the same or close absorption between the layers of the rolled paper stick 300. The absorption gradient between the layers of the rolled paper stick 300 can increase from the inner layer to the outer layer or decrease from the inner layer to the outer layer. This rolled paper stick 300 absorbs aromatic substances, such as liquid aromatic substance essences or essential oils or sesame oil. The outer layer absorbs more than the inner layer and can smoothly fill the fragrance to the inner layer or release the aromatics without hindrance. The product manufacturing process can be efficient and rapid.

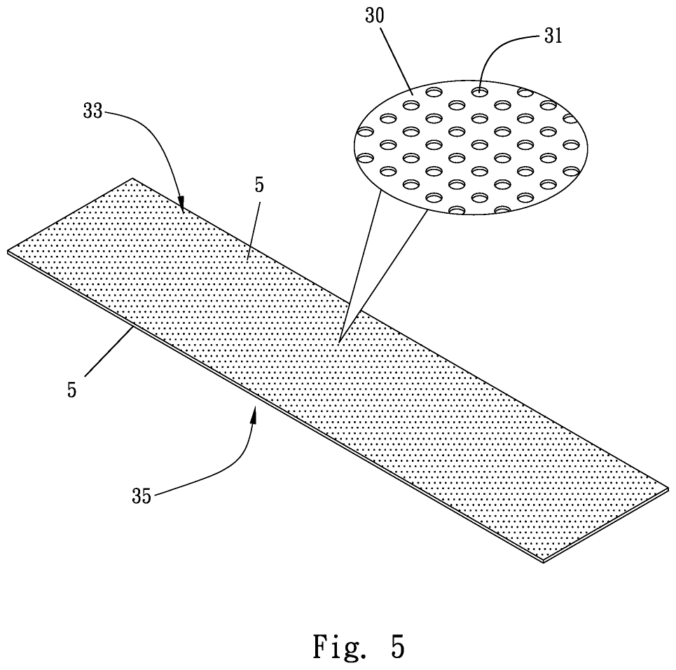

Referring to FIGS. 5-7, in one embodiment of the present invention, the breathable film 5 is a peelable, breathable film, whereby, at a later stage of the use of the diffuser product, the evaporation rate of the aromatic substance is weakened, and at least a part of the breathable film 5 is peeled off from the outer side of the absorbent paper structure component 3 to enhance the release rate of the aromatic substance.

In some embodiments of the present invention, the absorbent paper structure component 3 can be, but not limited to, a paper stick that can be a rolled paper stick 300 or a single structure paper stick made by casting. The single structure paper stick can be made of pulp or a pulp composition by casting.

In some embodiments of the present invention, the breathable film 5 is a multilayer film. In some embodiments, the breathable film 5 is, for example, a film of two, three or more layers bonded to the outer side of the absorbent paper structure component 3 which absorbs the aromatic substance, so that the breathable film 5 adjusts the release of the aromatic substance, such as volatile top notes, meddle notes, or base notes. Among them, the natural essential oils of the top notes have a strong diffusive force and volatilize quickly. The multiple layers of the breathable film 5 can be used to shield the volatile composition of this diffuser product in a waterproof and breathable manner to formulate and optimize the mixed release rate of various volatile compositions including top notes, middle notes and base notes. Through the multi-layer structure, the volatile top note is blocked to release slowly, and at the same time, other volatile combinations such as middle and base notes are released simultaneously in the space formed between the layers of the breathable film 5, or the volatile combinations are released over time in a staggered manner.

Referring to FIGS. 1-7, in some embodiments of the present invention, a diffuser product manufacturing method includes the steps as follows:

(a) Place an absorbent paper structure component 3 or absorbent paper 30 in an oven (not shown in the drawings, people skilled in the art can understand the application) and then operate the oven to bake the absorbent paper structure component 3 or absorbent paper 30 at about 60° C.-80° C. for about 15-25 minutes, and then remove the baked absorb paper structure component 3 or absorbent paper 30 from the oven.

(b) Use a container to fill at least one liquid aromatic substance in the absorbent paper structure component 3 or absorbent paper 30, enabling the liquid aromatic substance to be releasably remained in the pores 31 of the absorbent paper structure component 3 or absorbent paper 30.

(c) Remove the absorbent paper structure component 3 or absorbent paper 30 being filled with the liquid aromatic substance from the container for drying, and the dried absorbent paper structure component 3 or absorbent paper 30 is suitable for diffusing the aromatic substance in pores 31 into the atmosphere.

(d) Bond at least one breathable film 5 to the outer side of the dried absorbent paper structure component 3 or absorbent paper 30.

With this, the breathable film 5 can shield the absorbent paper structure component 3 or absorbent paper 30 in a waterproof and breathable manner and adjust the aromatic substance in the absorbent paper structure component 3 or absorbent paper 30 to contact with the outside air to further control the release rate of the aromatic molecules evaporated by the aromatic substance through the breathable film 5, and can also can control the outside moisture from penetrating into the inside of the absorbent paper structure component 3 or absorbent paper 30.

The breathable film 5 is a microporous film that is used to restrict the release of some of the aromatic substance to the outside while the micropores of the breathable film 5 allow some of the aromatic substance to gradually diffuse into the environmental space at a stable rate.

By adjusting the release rate of the appropriate amount of the volatile aromatic substance, it can delay the aroma diffusion time of the diffuser product, provide a stable and effective aroma to the space, and overcome the problem that the diffuser product consumes the aromatic substance too quickly in the early stage of use and the problem of insufficient concentration in the later stage.

The breathable film 5 is a waterproof and moisture-permeable microporous film. Therefore, the breathable film 5 not only can shield the absorbent paper structure component 3 in a waterproof and breathable manner and adjust the aromatic substance to contact with the outside air to further control the release rate of the aromatic molecules evaporated by the aromatic substance through the breathable film 5 but also can control the outside moisture from penetrating into the inside of the absorbent paper structure component 3 to maintain the quality of the absorbent paper structure component 3.

In some embodiments, the breathable film 5 can be bonded to the outer side of the absorbent paper structure component 3 or absorbent paper 30 by using an adhesive, starch, a reusable adhesive, a resin, a hot melt adhesive, a bonding agent, or any other adhesive material. Alternatively, ultrasonic bonding technology can be employed to bond the breathable film 5 to the outer side of the absorbent paper structure component 3 or absorbent paper 30.

Referring to FIGS. 1-3, the step of filling the at least one liquid aromatic substance into the absorbent paper structure component 3 or absorbent paper 30 can be implemented by: immersing the absorbent paper structure component 3 or absorbent paper 30 in a liquid aromatic substance in a container for a predetermined period of time so that the liquid aromatic substance can be releasably filled in the pores 31 of the absorbent paper structure component 3 or absorbent paper 30. In some embodiments, the liquid aromatic substance can be releasably filled into the pores 31 of the absorbent paper structure component 3 or absorbent paper 30 by means of contact, spraying, coating, pressurization, or aroma gas penetration.

Referring to FIGS. 1-3, in some embodiments of the present invention, the absorbent paper 30 is a planar paper material, and the liquid aromatic substance can be releasably filled into the pores 31 of the planar paper by means of coating, spraying or contacting. The pores 31 of the planar paper can releasably absorb the aromatic substance.

Referring to FIGS. 4-6, in one embodiment of the present invention, the step of bonding the breathable film to the outer side of the absorbent paper structure component 3 or absorbent paper 30 is implemented by bonding the breathable film 5 to at least one surface of the planar paper having absorbed therein the aromatic substance and then processing the breathable film bonded planar paper to form the desired absorbent paper structure component 3.

The surface of the planar paper of the absorbent paper structure component 3 to which the breathable film 5 is bonded is the outer side, and the opposite surface of the planar paper of the absorbent paper structure component 3 is the inner side. The inner side of the planar paper without the breathable film 5 is wrapped about a central axis to form a multi-layer rolled paper stick 300. The rolled paper stick 300 can be a solid or hollow shaft. The planar paper has a first surface 33 and a second surface 35. The first surface 33 and the second surface 35 have the same porosity or the same number of pores, forming an excellent absorption gradient. The breathable film 5 is bonded to the first surface 33.

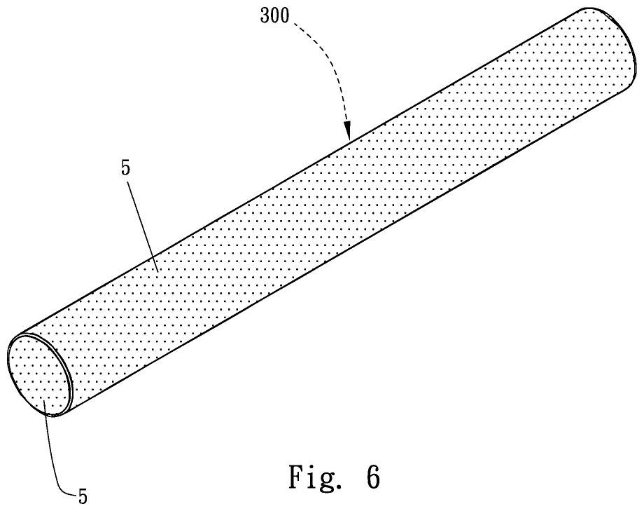

Referring to FIGS. 5 and 6, in one embodiment of the present invention, is the step pf bonding the breathable film 5 to the outer side of the dried absorbent paper structure component 3 or absorbent paper 30, the absorbent paper 30 is a planar paper material, the breathable film 5 is bonded to two opposite surfaces of the planar paper material that is filled with the aromatic substance. The planar paper is wrapped about a central axis to form a multi-layer rolled paper stick 300. The rolled paper stick 300 can be a solid or hollow shaft. The planar paper has a first surface 33 and a second surface 35. The first surface 33 and the second surface 35 have the same porosity or the same number of pores, forming an excellent absorption gradient. The breathable film 5 is bonded to the first surface 33 and the second surface 35.

Referring to FIGS. 1-5, in some embodiments of the present invention, the absorbent paper structure component 3 can be, but is not limited to, a paper roll, a paper stick, a paper piece, a paper block, a two-dimensional paper product, a three-dimensional paper product or a geometrically shaped absorbent paper structure component. The absorbent paper used has a first surface 33 and a second surface 35. The first surface 33 and the second surface 35 have the same porosity, the same number of pores.

In some embodiments of the present invention, the absorbent paper structure component 3 is selectively dried for a period of 15, 16, 17, 18, 19, 20, 21, 22, 23, 24 or 25 minutes.

In some embodiments of the present invention, the absorbent paper structure component 3 is selectively dried at 60, 61, 62, 63, 64, 65, 66, 67, 68, 69, 70, 71, 72, 73, 74, 75, 76, 77, 78, 79 or 80° C.

Referring to FIGS. 1-3, in some embodiments of the present invention, the absorbent paper structure component 3 is immersed in the liquid aromatic substance for a period of time about 30 seconds to 20 hours, for example, a 0.5×23 or 0.5×16 cm rolled paper stick 300 with the same inner and outer pores absorbs the aromatic substance from outside to inside, such as between 30 seconds or 1 minute to 10 minutes or tens of minutes, or one hour to several hours or several days. The immersing time is determined according to the parameters such as porosity or aroma concentration or pressure or absorption gradient, etc.

In one embodiment of the present invention, the breathable film 5 is a multi-layer breathable film. The multi-layer breathable film 5 is a waterproof and breathable laminate that shields the volatile composition of the diffuser product to formulate and optimize the mixed release rate of multiple volatile compositions including top, middle and base fragrances. The diffuser product slows the release of the top notes through the ventilation between the multi-layer structures, and at the same time, it is used to generate other volatile and other volatile composition substances, which are simultaneously released in the space formed between the multi-layer breathable film 5, or the volatile composition substances are released over time with different odors in a staggered manner. In some embodiments, the multi-layer breathable film 5 have micropores with the same or different porosity, or a gradient of release fragrance is formed between the multiple layers of the multi-layer breathable adjustment films 5 from the inside to the outside or from the outside to the inside to adjust various releases of the fragrance.

Referring to FIGS. 4 and 5, in some embodiments of the present invention, the breathable film 5 preferably includes all these microporous films such as polypropylene, polyethylene, ultra-high molecular weight polyethylene, polytetrafluoroethylene, or containing compounds that form pores, such as foaming agents. In some embodiments, the breathable film 5 is, for example, thermoplastic elastomer TPU (Thermoplastic Urethane), polytetrafluoroethylene TPFE (Polytetrafluoroethylene), which is waterproof and moisture-permeable.

Referring to FIGS. 4 and 5, in one embodiment of the present invention, the breathable film 5 is bonded to the outer side of the absorbent paper structure component 3 or absorbent paper 30. In some embodiments, the breathable film 5 can be attached or adsorbed or coated on the outer side of the absorbent paper structure component 3 or absorbent paper 30. In some embodiments, the breathable film 5 can be bonded to the outer side of the absorbent paper structure component 3 or absorbent paper 30 by using adhesives, starch, reusable adhesives, resins, hot melt adhesives, or other bonding materials. Alternatively, ultrasonic bonding technology can be employed to bond the breathable film 5 to the outer side of the absorbent paper structure component 3 or absorbent paper 30.

Referring to FIGS. 1-3, in one embodiment of the present invention, the diffuser product comprises a rolled paper stick 300. The rolled paper stick 300 is a multilayer structure formed by wrapping a planar absorbent paper around a central axis. The rolled paper stick 300 can be a solid or hollow shaft. The planar absorbent paper has a first surface 33 and a second surface 35. The first surface 33 and the second surface 35 have the same porosity and the same number of pores. The pores 31 are distributed in the rolled paper stick 300, and tiny air channels are formed between the pores 31 to produce capillary effects.

The rolled paper stick 300 is baked at 60° C.-80° C. for about 15-25 minutes to a dried state. After the rolled paper stick 300 is dried, the pores 31 have high saturation absorptivity, and the aromatic substance loading amount of the rolled paper stick 300 can be greatly increased.

At least one aromatic substance is releasably filled in the pores 31 of the dry state rolled paper stick 300. With this, each layer of the rolled paper stick 300 has the same porosity, forming an excellent absorption gradient or release gradient.

In the production process of the rolled paper stick 300, the rolled paper stick 300 is immersed in the liquid aromatic substance or disposed close to the liquid aromatic substance to absorb the liquid fluid by a capillary absorption effect. It has an excellent absorption rate and can effectively absorb the aromatic substance and fill it into the pores 31 of the absorbent paper structure component 3. The rolled paper stick 300 effectively conveys the filled aromatic substance in contact with outside air to dissipate the aromatic substance into the environmental space.

In some embodiments of the present invention, the aromatic substance is a liquid aromatic substance. The liquid aromatic substance further includes, but is not limited to, fragrances, fragrance oils, fragrance compounds, fragrance compositions, aromatherapy compounds or other volatile compositions, top notes, middle notes, or base fragrances. It is not limited to a single substance or composition or solvent of essential oils, aromatic essential oils, flavors, fragrances, dew, etc.

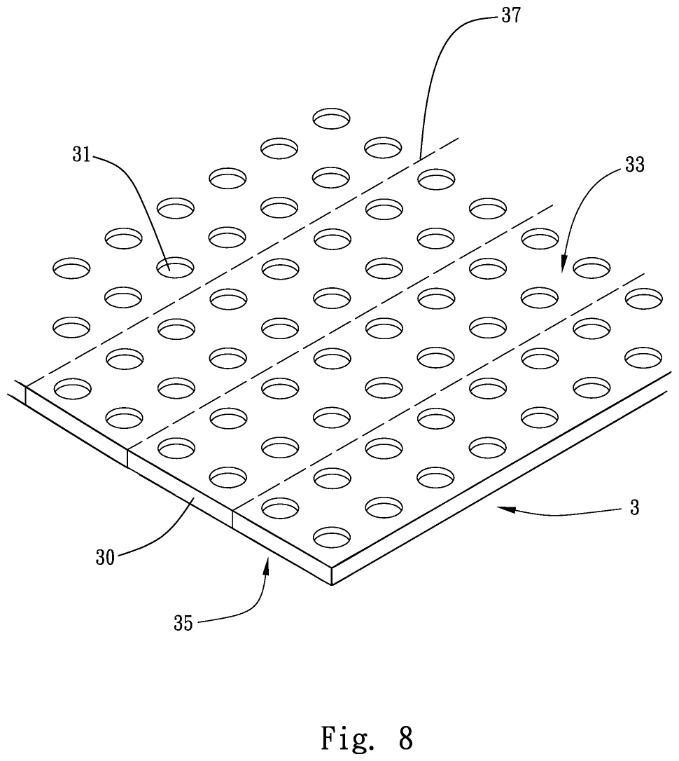

Referring to FIGS. 1-3 and FIG. 8, in one embodiment of the present invention, the planar paper 30 has a plurality of tear lines 37. The plurality of tear lines 37 are spaced in parallel and spaced apart from each other in the axial direction of the winding axis of the planar paper 30 of the multilayer structure of the rolled paper stick 300. With this, the user can peel off one tear line 37 of the rolled paper stick 300 to separate a small piece of paper from the rolled paper stick 300 into a single aromatic paper for independent application. Or in the late stage of the rolled paper stick 300, when the evaporation rate of the aromatic substance is weakened, the paper on the outside of the rolled paper stick 300 is peeled off through one of the tear lines 37 to expose the effective aromatic substance remaining inside the rolled paper stick 300, thereby prolonging the fragrance release time of the rolled paper stick 300.

What the invention claimed is:

1. A diffuser product, comprising:
an absorbent paper structure component, said absorbent paper structure component comprising an absorbent paper, said absorbent paper comprising a plurality of pores and a plurality of tiny air channels formed between said pores for creating capillary effects;
at least one aromatic substance releasably filled in said pores of said absorbent paper of said absorbent paper structure component; and
at least one breathable film bonded to an outer side of said absorbent paper or said absorbent paper structure component, said at least one breathable film having a plurality of micropores that are waterproof and breathable;
wherein said at least one breathable film shield said absorbent paper structure component in a waterproof and breathable manner, and is used to adjust said at least one aromatic substance to contact with the outside air, thereby controlling the release rate of aromatic molecules evaporated by said at least one aromatic substance through said breathable film and also controlling the outside moisture from penetrating into the inside of said absorbent paper structure component.

2. The diffuser product as claimed in claim 1, wherein said at least one breathable film is selected made from thermoplastic polyurethane (TPU), polytetrafluoroethylene (TPFE), polyethylene, polypropylene, or ultra-high molecular weight polyethylene.

3. The diffuser product as claimed in claim 1, wherein said at least one breathable film is selectively bonded to at least a part or all or the end of the outer side of said absorbent paper or said absorbent paper structure component, or bonded with the outer side of said absorbent paper structure component at intervals.

4. The diffuser product as claimed in claim 3, wherein said absorbent paper structure component is a paper stick and said paper stick is selectively a rolled paper stick or a single structure paper stick made by casting.

5. The diffuser product as claimed in claim 3, wherein said absorbent paper structure component is a rolled paper stick, said rolled paper stick being a multilayer structure formed by wrapping a planar absorbent paper around a central axis, said rolled paper stick being selectively a solid or hollow shaft, said planar paper having a first surface and a second surface, said first surface and said second surface having the same porosity.

6. The diffuser product as claimed in claim 4, wherein said breathable film is a peelable, breathable film.

7. The diffuser product as claimed in claim 1, wherein said at least one aromatic substance includes an aromatic odor composition, a volatile substance, or a liquid aromatic substance, such as a fragrance or fragrance composition, an aromatherapy compound, a water-based fragrance or a natural oil that is transmittable to the olfactory system and has one or more aromatic characteristics, such as fragrance ingredients such as top notes, middle notes or base notes.

8. The diffuser product as claimed in claim 1, wherein said at least one breathable film has multiple layers.

9. A diffuser product manufacturing method, comprising the steps of:
(a) placing an absorbent paper structure component or absorbent paper in an oven and then operating said oven to bake said absorbent paper structure component or said absorbent paper at about 60° C.-80° C. for about 15-25 minutes, and then removing the baked said absorb paper structure component or said absorbent paper from said oven;
(b) using a container to fill at least one liquid aromatic substance into said absorbent paper structure component or said absorbent paper for enabling said at least one liquid aromatic substance to be releasably remained in pores of said absorbent paper structure component or said absorbent paper;
(c) removing said absorbent paper structure component or said absorbent paper being filled with said at least one liquid aromatic substance from said container for drying, and the dried said absorbent paper structure component or said absorbent paper being suitable for diffusing said at least one aromatic substance into the atmosphere; and
(d) bonding at least one breathable film to the outer side of the dried said absorbent paper structure component or said absorbent paper.

10. The diffuser product manufacturing method as claimed in claim 9, wherein the step of filling said at least one liquid aromatic substance into said absorbent paper structure component or said absorbent paper is implemented by: immersing said absorbent paper structure component or said absorbent paper in at least one liquid aromatic substance in a container for a predetermined period of time so that said at least one liquid aromatic substance is releasably filled in pores of said absorbent paper structure component or said absorbent paper

11. The diffuser product manufacturing method as claimed in claim 10, wherein said absorbent paper structure component or said absorbent paper is immersed in said at least one liquid aromatic substance in said container for 30 seconds to 20 hours.

12. The diffuser product manufacturing method as claimed in claim 9, wherein in the step of filling said at least one liquid aromatic substance into said absorbent paper structure component or said absorbent paper, said absorbent paper is a planar paper material having pores therein, and said at least one liquid aromatic substance is releasably filled into the pores of said planar paper material by means of coating, spraying or contacting.

13. The diffuser product manufacturing method as claimed in claim 9, wherein in the step of bonding said at least one breathable film to the outer side of the dried said absorbent paper structure component or said absorbent paper, said absorbent paper is a planar paper, said at least one breathable film is bonded to one surface of said absorbent paper, the opposite surface of said absorbent paper is the inner side, and said inner side of said absorbent paper is wrapped about a central axis to form a multi-layer rolled paper stick, said rolled paper stick being selectively a solid or hollow shaft, said absorbent paper having a first surface and a second surface, said first surface and said second surface having the same porosity.

14. The diffuser product manufacturing method as claimed in claim 13, wherein in the step of bonding said at least one breathable film to the outer side of the dried said absorbent paper structure component or said absorbent paper, said absorbent paper is a planar paper material, and said at least one breathable film is bonded to two opposite surfaces of said planar paper material after said planar paper material is filled with said at least one aromatic substance.

15. The diffuser product manufacturing method as claimed in claim 9, wherein said absorbent paper structure component is selectively dried for a period of 15, 16, 17, 18, 19, 20, 21, 22, 23, 24 or 25 minutes.

16. The diffuser product manufacturing method as claimed in claim 9, wherein said absorbent paper structure component is selectively dried at 60, 61, 62, 63, 64, 65, 66, 67, 68, 69, 70, 71, 72, 73, 74, 75, 76, 77, 78, 79 or 80° C.

17. A diffuser product, comprising:
a rolled paper stick, said rolled paper stick being a multilayer structure formed by wrapping a planar absorbent paper around a central axis, said rolled paper stick being selectively a solid or hollow shaft, said planar paper having a first surface and a second surface, a plurality of pores formed on each of said first surface and said second surface and a plurality of micro air channels formed on each of said first surface and said second surface between said pores for creating capillary effects, said first surface and said second surface having the same porosity, said rolled paper stick being dried so that said pores have high saturation absorptivity; and
at least one aromatic substance releasably filled in said pores of the dried said rolled paper stick.

18. The diffuser product as claimed in claim 17, wherein each of said at least one aromatic substance is a liquid aromatic substance.

19. The diffuser product as claimed in claim 17, wherein said planar absorbent paper has a plurality of tear lines.
